(19) United States
(12) Patent Application Publication
(10) Pub. No.: US 2025/0123254 A1
(43) Pub. Date: Apr. 17, 2025

(54) METHODS FOR MUTANT CEREAL CROP LINES

(71) Applicants: BASF SE, Ludwigshafen (DE); Commonwealth Scientific and Industrial Research Organization, Australian Capital Territory (AU)

(72) Inventors: Claus Frohberg, Gent (BE); Ralf-Christian Schmidt, Gent (BE); Anthony Gerard Condon, Yarralumla (AU); Jose Barrero Sanchez, Acton (AU)

(21) Appl. No.: 18/567,434

(22) PCT Filed: Jun. 7, 2022
(86) PCT No.: PCT/EP2022/065368

§ 371 Date: Dec. 6, 2023

(30) Foreign Application Priority Data
Jun. 11, 2021 (EP) 21179057.1

(51) Int. Cl. A01G 22/20; G01N 33/02

(52) U.S. Cl. CPC G01N 33/025

(57) ABSTRACT

Methods are provided for identifying and selecting mutant cereal crop lines having high biomass values within a period corresponding to Zadok's growth stages of from about Z30 to Z39; for identifying and selecting mutant cereal crop lines having potential for increased yield; and for generating a grain yield prediction in mutant cereal crop lines.

### FIELD

The present invention relates to methods for identifying and selecting mutant cereal crop lines having high biomass values within a period corresponding to Zadok's growth stages of from about Z30 to Z39; methods for identifying and selecting mutant cereal crop lines having potential for increased yield; and methods for generating a grain yield prediction in mutant cereal crop lines.

### BACKGROUND

Developing cereal crops that produce increased yield is an important step to securing human food supply and meeting the nutritional demands of rapid population growth. Development of new crop lines and varieties, including identifying lines with favorable traits such as high yield, is a lengthy and resource-consuming process. Most available processes for determining yield of new mutant lines are post-harvest methods. Therefore, large numbers of different mutant lines need to be provided with growing resources and then harvested to find candidates for further breeding and development. Harvest and processing of low or mid yield populations can result in high cost and time input, with breeders only learning after such input that a particular population was not worth harvesting. Additionally, candidates with good yield as measured by post-harvest procedures in controlled growing conditions such as greenhouses or test plots often fail to show similar yield once grown in the field. Pre-harvest methods for predicting yield have thus far been of limited accuracy or fail to sufficiently reduce resource use by providing yield predictions in late development stages just before harvest when significant resources have already been used.

### SUMMARY OF THE INVENTION

Thus, there remains a need for improved and earlier yield insights among mutant cereal crop lines. There is a need for new methods for identifying and selecting mutant lines having increased yield or potential for increased yield or predicting grain yield, particularly during earlier growth stages. There is also a need for methods that can provide high throughput screening of mutant lines for potential yield performance, particularly during earlier growth stages. The methods provided herein address these needs.

In one aspect, provided herein is a method comprising:
growing one or more mutant cereal crop lines;
measuring or estimating at least one biomass value for each mutant line within a period corresponding to Zadok's growth stages of from about Z30 to about Z39;
identifying one or more mutant lines having at least one high biomass value within the period, wherein high biomass value is selected from:
   a biomass value that is within a highest portion of biomass values for a plurality of mutant lines, wherein the biomass values for each of the plurality of mutant lines are measured or estimated at a same or similar growth stage within the period, wherein the plurality of mutant lines includes the one or more mutant cereal crop lines and wherein the plurality of mutant lines is a mutant population, or
   a biomass value exceeding a threshold biomass value, wherein the threshold biomass value is obtained from one or more members of a mutant population containing the one or more mutant cereal crop lines or is obtained from a non-mutagenized cereal crop line corresponding to the one or more mutant cereal crop lines.

In some embodiments, the method can optionally include one or more of the following features. The method can further comprise selecting one or more of the one or more mutant lines having a high biomass value for further use. Further use can be selected from future breeding, genotyping, yield trialing, harvest, genetic mapping, or combinations thereof. The highest portion of early biomass values for a plurality of mutant lines can be selected from the highest 90%, highest 80%, highest 70%, highest 60%, highest 50%, highest 40%, highest 30%, highest 20%, highest 10%, or highest 5% of the early biomass values. The threshold biomass value can be a biomass value of a reference crop line measured or estimated at a same or similar growth stage within the period, or an average or median biomass value determined from a plurality of mutant lines measured or estimated at a same or similar growth stage within the period. The period can be a period corresponding to Zadok's growth stages of from about Z33 to about Z38. The period can be a period corresponding to Zadok's growth stages of from about Z34 to about Z36. Measuring or estimating at least one biomass value for each mutant line can comprise measuring or estimating at least one biomass value for a portion of a row, a plot, or a farmer's field containing plants of the mutant line. Measuring or estimating at least one biomass value for each mutant line can comprise measuring or estimating at least one biomass value for a portion of a test plot, wherein the test plot contains a single mutant line. Measuring or estimating at least one biomass value for each mutant line can comprise measuring or estimating at least one biomass value for a portion of a farmer's field. Measuring or estimating at least one biomass value for each mutant line can comprise measuring or estimating at least one biomass value for a portion of a test row. The test row can be one of a plurality of interplanted rows, wherein each row independently contains plants from a single mutant line or reference line, and wherein each row contains a different mutant line or reference line than an adjacent row. The test row can be part of a row plot comprising a central portion containing one or more parallel adjacent test rows, wherein the central portion is flanked by at least one border row at each of two opposite sides of the central portion, wherein each of the at least one border rows are sown parallel to the test rows of the central portion. Each test row of the central portion can independently contain a mutant line and each border row can independently contain a reference line. Growing one or more mutant lines can comprise growing each mutant line in a single row. The single row can be sown with from about 40 to about 80 seeds. Measuring or estimating biomass can be performed by a destructive measurement method. The destructive measurement method can be biomass cuts. Measuring or estimating biomass can be performed by a non-destructive measurement or estimation method. The non-destructive measurement or estimation method is selected from a LiDAR-based method for biomass estimation and a NDVI-based method for biomass estimation. Measuring or estimating biomass can be performed by a LIDAR-based method for biomass estimation. The LiDAR-based method for biomass estimation can be selected from a vox-based estimation method and a profile-based estimation method. The method can further comprise generating at least one of the one or more mutant cereal crop lines by mutagenesis. The mutagenesis can be selected from chemical mutagenesis, physical mutagenesis, mutagenesis by genome editing, mutagenesis by transgenesis, or combinations thereof. The cereal crop can be selected from wheat, barley, sorghum, rice, rye, oats, or triticale. The method can further comprise identifying one or more traits other than biomass in each of the one or more mutant lines, and, optionally, can further comprise selecting one or more of the one or more mutant lines having a high biomass value for further use based on the identifying one or more traits other than biomass. The method can be a method for high throughput screening of mutant cereal crop lines, wherein the mutant lines are screened for further use or processing based on the biomass value.

In another aspect, provided herein is a method comprising:
growing one or more mutant cereal crop lines;
measuring or estimating at least one biomass value for each mutant line within a period corresponding to Zadok's growth stages of from about Z30 to about Z39;
generating a grain yield prediction for each mutant line based on the at least one biomass value for the mutant line.

Generating a grain yield prediction for each mutant line can comprise at least one selected from: ordering the biomass values for each of at least two mutant lines belonging to a mutant population in an ascending or descending order of biomass values and assigning a relative yield prediction to each mutant line corresponding to a position of the mutant's at least one biomass value in the order of biomass values, wherein the biomass values for each mutant line is measured or estimated at a same or similar growth stage within the period; comparing the at least one biomass value for each mutant line with biomass values for a plurality of mutant lines, wherein the biomass values for each of the plurality of mutant lines are biomass values for each, and generating a yield prediction based on a relation of the mutant's at least one biomass value to the biomass values for each of the plurality of mutant lines, wherein the plurality of mutant lines includes the one or more mutant cereal crop lines and wherein the plurality of mutant lines is a mutant population; or determining a comparison for each mutant's biomass value against a threshold biomass value and generating a yield prediction based on each mutant's comparison, wherein the threshold biomass value is obtained from one or more members of a mutant population containing the one or more mutant cereal crop lines or is obtained from a non-mutagenized cereal crop line corresponding to the one or more mutant cereal crop lines. In some embodiments, the method can optionally include one or more of the following features. The threshold biomass value can be a biomass value of a reference crop line measured or estimated at a same or similar growth stage within the period, or an average or median biomass value determined from a plurality of mutant lines measured or estimated at a same or similar growth stage within the period. The method can further comprise selecting one or more of the one or more mutant lines having a high biomass value for further use. The further use can be selected from future breeding, genotyping, yield trialing, harvest, genetic mapping, or combinations thereof. The period can be a period corresponding to Zadok's growth stages of from about Z33 to about Z38. The period can be a period corresponding to Zadok's growth stages of from about Z34 to about Z36. Measuring or estimating at least one biomass value for each mutant line can comprise measuring or estimating at least one biomass value for a portion of a row, a plot, or a farmer's field containing plants of the mutant line, and, optionally, the yield prediction can be selected from a row yield prediction, a plot yield prediction, or a farmer's field yield prediction. Measuring or estimating at least one biomass value for each mutant line can comprise measuring or estimating at least one biomass value for a portion of a test plot, wherein the test plot contains a single mutant line. Measuring or estimating at least one biomass value for each mutant line can comprise measuring or estimating at least one biomass value for a portion of a farmer's field. Measuring or estimating at least one biomass value for each mutant line can comprise measuring or estimating at least one biomass value for a portion of a test row. The test row can be one of a plurality of interplanted rows, wherein each row independently contains plants from a single mutant line or reference line, and wherein each row contains a different mutant line or reference line than an adjacent row. The test row can be part of a row plot comprising a central portion containing one or more parallel adjacent test rows, wherein the central portion is flanked by at least one border row at each of two opposite sides of the central portion, wherein each of the at least one border rows are sown parallel to the test rows of the central portion. Each test row of the central portion can independently contain a mutant line and each border row can independently contain a reference line. Growing one or more mutant lines can comprise growing each mutant line in a single row. The single row can be sown with from about 40 to about 80 seeds. Measuring or estimating biomass can be performed by a destructive measurement method. The destructive measurement method can be biomass cuts. Measuring or estimating biomass can be performed by a non-destructive measurement or estimation method. The non-destructive measurement or estimation method is selected from a LIDAR-based method for biomass estimation and a NDVI-based method for biomass estimation. Measuring or estimating biomass can be performed by a LIDAR-based method for biomass estimation. The LiDAR-based method for biomass estimation can be selected from a vox-based estimation method and a profile-based estimation method. The method can further comprise generating at least one of the one or more mutant cereal crop lines by mutagenesis. The mutagenesis can be selected from chemical mutagenesis, physical mutagenesis, mutagenesis by genome editing, mutagenesis by transgenesis, or combinations thereof. The cereal crop can be selected from wheat, barley, sorghum, rice, rye, oats, or triticale. The method can further comprise identifying one or more traits other than biomass in each of the one or more mutant lines, and, optionally, can further comprise selecting one or more of the one or more mutant lines having a high biomass value for further use based on the identifying one or more traits other than biomass. The method can be a method for high throughput screening of mutant cereal crop lines, wherein the mutant lines are screened for further use or processing based on the biomass value.

In another aspect, described herein are mutant cereal lines produced by the methods described herein.

The methods described herein may provide several advantages. First, the inventors have surprisingly found that measurements or estimation of biomass in a mutant population of cereal crop lines within a period corresponding to Zadok's growth stages of from about Z30 to about Z39 are strongly correlated to or indicative of yield, allowing for an early, pre-harvest tool for estimating or predicting yield, and for methods of identifying or selecting mutant cereal crop lines. Second, by allowing for early identification, selection, yield prediction, or screening of candidate mutant lines exhibiting increased yield or having a potential or probability for increased yield, methods described herein may, in some embodiments, allow for in-season identification, selection, yield prediction, or screening of candidate mutant lines, thus allowing researchers and breeders to focus resources on fewer mutant lines with greater success. The inventors have surprisingly found that measuring or estimating biomass within a period corresponding to Zadok's growth stages Z30 to Z39 can provide insights into identifying, selecting, or screening mutant cereal crop lines that are likely to display increased yield, for example, with respect to the corresponding non-mutagenized cereal crop line. Because methods described herein can be performed during growth stages corresponding to Zadok's growth stages Z30 to Z39, specific mutant lines of interest can be selected for harvest or non-harvest, or for other activities such as genotyping or further breeding, within a sufficient amount of time to decrease plant growth support and harvest resources, and to select and prepare lines for upcoming growing seasons.

Third, methods described herein can, in some embodiments, provide insights that are applicable to larger plots and fields, even when the methods are performed on plants grown in single rows, in small test plots, and the like. For example, in some embodiments, methods described herein can provide, from measurement or estimation of biomass on a test row, identification of mutant lines having high biomass within a period corresponding to Zadok's growth stages Z30 to Z39, and can, in some embodiments, use this identification to accurately predict corresponding plot yield. The inventors have surprisingly found that row biomass within a period corresponding to Zadok's growth stages Z30 to Z39 has a better correlation with plot yield than attempts at correlating row yield to plot yield.

Fourth, methods described herein can, in some embodiments, be performed on single rows, containers, controlled environments and the like. The single row methods can provide insights applicable on larger plots and fields. This may allow breeders to use fewer resources. It also advantageously provides methods for identification, selection, yield prediction, or screening of candidate mutant lines even where only small amounts of seed are available. In addition, some embodiments of single row methods can allow a relatively high number of lines to be tested on a relatively small land or plot area.

Fifth, methods described herein can, in some embodiments, provide beneficial timing for farmers, breeders, researchers, or other users of the methods. For example, in typical breeding activities, harvest and data analysis usually need to be completed before lines can be selected for the next season. This can present challenges in harvesting and processing the data in time to make the selections and subsequent preparations prior to the next growing season. In some embodiments of the methods described herein, the selection of desired lines can occur before harvest, allowing only lines for the next season to be harvested where desired. Additionally, in some embodiments, because selection has already been made prior to harvest, there may be no additional post-harvest analysis needed. Thus some embodiments described herein can allow farmers, breeders, researchers, and other users more time to prepare for the next season. This can also in turn provide more flexibility for time of planting in the next season as seeds are available for sowing.

Sixth, methods described herein can, in some embodiments, provide an opportunity to steer parallel running crossing or breeding activities because selection of desired lines can be made early in the season and can, in some embodiments allow parallel breeding and crossing that is restricted to the best performing lines. As only a small number of seeds are needed in some embodiments of the methods described herein (e.g. row or single row), this kind of selection or screening can be done at a very early stage of a breeding process, thus making selection more efficient.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

For the terms “for example” and “such as,” and grammatical equivalences thereof, the phrase “and without limitation” is understood to follow unless explicitly stated otherwise. As used herein, the term “about” is meant to account for variations due to experimental error. As used herein, the singular forms “a,” “an,” and “the” are used interchangeably and include plural referents unless the context clearly dictates otherwise.

The details of one or more implementations of the subject matter of this disclosure are set forth in the accompanying drawings and the description. Other features, aspects, and advantages of the subject matter will become apparent from the description, the drawings, and the claims.

### BRIEF DESCRIPTION OF THE DRAWINGS

FIG. 1 is a graph showing correlation between row yield and row biomass at approximately Zadok's growth stage Z38 (Date 2, Season 1) for all mutant lines (r²=0.461) is described in Example 1.

FIG. 2 is a top plan view of a diagram of an exemplary test plot.

FIG. 3 is a graph showing correlation between row yield and row biomass at approximately Zadok's growth stage 35 (Date 2, Season 2) for all mutant lines (r²=0.422), as described in Example 1.

### DETAILED DESCRIPTION

Described herein are methods relating to cereal crop lines and mutant cereal crop lines. Cereal crops applicable in the methods described herein can include, for example, wheat, barley, sorghum, rice, rye, oats, or triticale.

As used herein, “mutant cereal crop line,” “mutant line,” and “mutant” refer to a crop line (e.g., a cereal crop line, such as a wheat crop line) bearing at least one mutation in its genome resulting from mutagenesis (e.g., as compared to a corresponding non-mutagenized cereal crop line). As used herein, “mutant population” or “population of mutant lines” and the like refers to a group or plurality of different mutant lines obtained from mutagenesis of a genetically and phenotypically homogenous cereal crop line, in which the propagatable mutagenized parts of that line have been kept separate and gone through at least one round of self-pollination in order to obtain different seed batches containing distinct fixed mutations. Mutant lines can be produced or generated by any form of mutagenesis. Exemplary non-limiting mutagenesis techniques include chemical mutagenesis, physical mutagenesis, mutagenesis by genome editing, mutagenesis by transgenesis, and combinations thereof.

As used herein, descriptions referring to rows, plots, or fields containing specific or single mutant lines are to be understood to include small or negligible amounts of contaminants such as weeds or other plant lines.

In one aspect, a method is provided comprising:
growing one or more mutant cereal crop lines;
measuring or estimating at least one biomass value for each mutant line within a period corresponding to Zadok's growth stages of from about Z30 to about Z39;
identifying one or more mutant lines having at least one high biomass value within the period, wherein high biomass value is selected from:
   a biomass value that is within a highest portion of biomass values for a plurality of mutant lines, wherein the biomass values for each of the plurality of mutant lines are measured or estimated at a same or similar growth stage within the period, wherein the plurality of mutant lines includes the one or more mutant cereal crop lines and wherein the plurality of mutant lines is a mutant population, or
   a biomass value exceeding a threshold biomass value, wherein the threshold biomass value is obtained from one or more members of a mutant population containing the one or more mutant cereal crop lines or is obtained from a non-mutagenized cereal crop line corresponding to the one or more mutant cereal crop lines.

In some embodiments, the at least one biomass value is measured or estimated within a period corresponding to a Zadok's growth stage (see, for example, Zadoks, J. C et al., (1974). Weed Research 14 (6): 415-421, which is incorporated herein in its entirety) of from about Z30 to about Z39. In some embodiments, the at least one biomass value is measured or estimated within a period corresponding to a Zadok's growth stage of from about Z33 to about Z38. In some embodiments, the at least one biomass value is measured or estimated within a period corresponding to a Zadok's growth stage of from about Z34 to about Z36. Biomass values measured or estimated at other growth stages can also be used for methods described herein, however, the inventors have surprisingly found a strong correlation of biomass to yield performance in the Zadok's growth stages of from about Z30 to about Z39, which can advantageously allow for early analysis and selection of mutant lines, including in-season selection. While reference is made herein to Zadok's growth stages, it is to be understood that the methods described herein could be performed with reference to other cereal growth staging scales, provided the time period within which the biomass values are obtained correspond to the same growth time periods as those described for the Zadok's staging scale.

In some embodiments, one or more biomass values are measured or estimated. In some embodiments where more than one biomass value is obtained within the period of Z30 to Z39 for a given mutant line, a single biomass value may be selected for each mutant in order to identify mutant lines having at least one high biomass value within a period corresponding to growth stages selected from Z30 to Z39. In some embodiments where more than one biomass value is obtained within the period of Z30 to Z39 for a given mutant line, a set of biomass values may be selected for each mutant in order to identify mutant lines having at least one high biomass value within a period corresponding to growth stages selected from Z30 to Z39. In some embodiments where more than one biomass value is obtained within the period of Z30 to Z39 for a given mutant line, an average or median biomass value may be calculated for each mutant in order to identify mutant lines having at least one high biomass value within a period corresponding to growth stages selected from Z30 to Z39.

In some embodiments, the estimated or measured biomass values can be compared within a plurality of biomass values for a plurality of mutant lines in order to identify mutant lines within a highest portion of biomass values for a plurality of mutant lines. Exemplary highest portions can include a highest 90%, highest 80%, highest 70%, highest 60%, highest 50%, highest 40%, highest 30%, highest 20%, highest 10%, or highest 5% of the early biomass values, each corresponding to a mutant line. A highest portion of biomass values can be any portion selected by the breeder or researcher as a target portion, and can be adapted by those skilled in the art. For example, in some instances, a breeder may have limited resources and therefore may choose to select only a small highest portion, for example, a highest 10% of early biomass values, each corresponding to a mutant line.

In some embodiments, the estimated or measured biomass values can be compared against a threshold biomass value in order to identify mutant lines having a biomass value exceeding the threshold biomass value. The threshold biomass value is obtained from one or more members of a mutant population containing the one or more mutant cereal crop lines or is obtained from a non-mutagenized cereal crop line corresponding to the one or more mutant cereal crop lines. In some embodiments, a threshold biomass value can include an average biomass value or a reference biomass value, such as, but not limited to a control value or a value obtained from a mutant's corresponding non-mutagenized line. In some embodiments, a threshold biomass value can be a biomass value of a reference crop line. A reference line or reference crop line can be a control or a corresponding non-mutagenized cereal crop line or any other line. For example, a reference line for a mutant population can be the corresponding non-mutagenized cereal crop line or a line from a seed batch from which the mutagenized seed was created. In some embodiments, the biomass values being compared were measured or estimated at a same or similar growth stage within the period of growth stages Z30 to Z39. Same growth stages can, in some embodiments, indicate the same Z stage, for example, Z35. Similar growth stages can, in some embodiments, indicate an adjacent growth stage. For example, biomass values measured in adjacent growth stages can include a particular Z stage and the next or prior Z stage. In some embodiments, similar growth stages can mean a latest portion of one Z stage and an early portion of the next sequential Z stage.

In some embodiments, the threshold or average values indicate one or more biomass values within a period from Z30 to Z39 of a corresponding non-mutagenized cereal crop line, or another target line for which yield higher than the target line is sought. For example, a breeder or researcher may create mutants of an initial line, and seek to identify mutant lines that exceed the biomass values within a period from Z30 to Z39 for the initial line.

In some embodiments, the measuring or estimating biomass is performed on mutant crop lines sown in rows or row plots, in plots or test plots, or in fields such as a farmer's field. Advantageously, in some embodiments, the measurements or estimation does not need to be performed on whole rows, plots, or fields, and can instead optionally be performed on a portion of a row, plot, or field to reduce time and resource use. In some embodiments, the methods described herein can also be performed on lines sown in containers, controlled environments, and the like.

In some embodiments, the measuring or estimating can comprise measuring or estimating at least one biomass value for a portion of a test plot. In some embodiments, the test plot can contain a single mutant line. Test plots can be of any desired size. In some embodiments, the measuring or estimating can comprise measuring or estimating at least one biomass value for a portion of a farmer's field.

In some embodiments, the measuring or estimating can comprise measuring or estimating at least one biomass value for a portion of a test row. In some embodiments, a test row can be a single row. For example, a single row containing a single mutant line can be sown and one or more biomass values can be measured or estimated during a period within growth stages Z30 to Z39. In some embodiments the at least one biomass value can then be compared against a reference biomass value or set of values. In some embodiments, the test row can be a single row within a plurality of interplanted rows, wherein each row independently contains plants from a single mutant line or reference line, and wherein each row contains a different mutant line or reference line than an adjacent row. For example, the test row can be a row in a row plot. Advantageously, rows can be sown with small amounts of seed, for example from about 40 to about 80 seeds per row. This can beneficially allow analysis and screening for yield or yield potential or high biomass within a period of from Z30 to Z39 even when only small amounts of seed are available for a given mutant line.

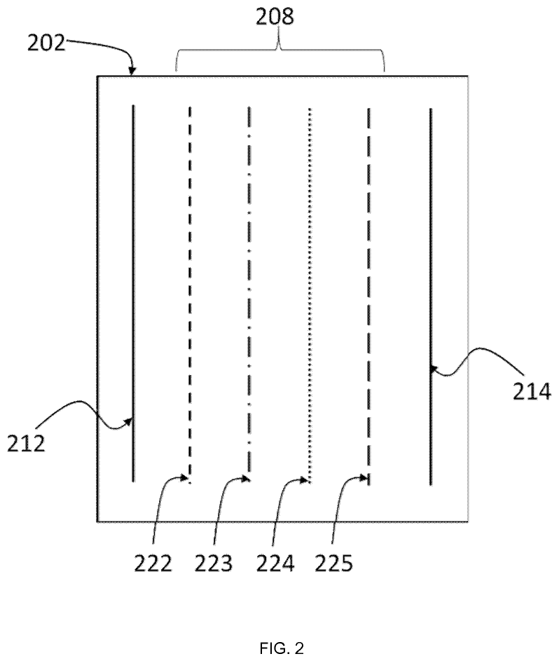

Referring to FIG. 2, a top plan view of an exemplary row plot 202 is shown. The row plot 202 can comprise a central portion 208 containing one or more parallel adjacent test rows 222, 223, 224, and 225, each containing a single mutant line (for example, row 222 containing a first mutant line, row 223 containing a second mutant line, row 224 containing a third mutant line, row 225 containing a fourth mutant line, and so on). Any number of rows may be included in the central portion 208 depending on the study design. In some embodiments, the central portion 208 can contain from 1 to 10 rows. In some embodiments, central portion 208 can optionally be flanked by two border rows, e.g., a first border row 212 and a second border row 214. The first border row 212 and second border row 214 are each independently positioned at an opposite side of the central portion 208 and sown parallel to test rows 222, 223, 224, and 225 of the central portion 208. In some embodiments, each test row of the central portion 208 can independently contain a same or different mutant line. In some embodiments, each border row 212 and 214 can independently contain a reference line.

Measuring or estimating biomass within a period corresponding to a Zadok's growth stage of between Z30 and Z39 can be performed by any biomass measurement or estimation method. Such measurement or estimation methods can be destructive or non-destructive. Non-destructive methods can advantageously allow for further uses of the individual plants that require continued growing, such as harvest. Exemplary destructive methods for measuring or estimating biomass include, but are not limited to, biomass cuts. Exemplary non-destructive methods for measuring or estimating biomass include, but are not limited to, using remote sensing methods such as NDVI or LiDAR measurements to estimate biomass, such as those described in Jimenez-Berni Jose A., et al., Front. Plant Sci., 27 Feb. 2018, Vol. 9, Art. 237. As another non-limiting example, measurement or estimation of ground cover can be used to estimate biomass, or can be used in place of biomass estimations or measurement in some embodiments of the methods described herein.

In another aspect, a method is provided herein comprising:
growing one or more mutant cereal crop lines;
measuring or estimating at least one biomass value for each mutant line within a period corresponding to Zadok's growth stages of from about Z30 to about Z39;
generating a grain yield prediction for each mutant line based on the at least one biomass value for the mutant line.

Generating a grain yield prediction for each mutant line can comprise at least one selected from: ordering the biomass values for each of at least two mutant lines belonging to a mutant population in an ascending or descending order of biomass values and assigning a relative yield prediction to each mutant line corresponding to a position of the mutant's at least one biomass value in the order of biomass values, wherein the biomass values for each mutant line is measured or estimated at a same or similar growth stage within the period; comparing the at least one biomass value for each mutant line with biomass values for a plurality of mutant lines, wherein the biomass values for each of the plurality of mutant lines are biomass values for each, and generating a yield prediction based on a relation of the mutant's at least one biomass value to the biomass values for each of the plurality of mutant lines, wherein the plurality of mutant lines includes the one or more mutant cereal crop lines and wherein the plurality of mutant lines is a mutant population; or determining a comparison for each mutant's biomass value against a threshold biomass value and generating a yield prediction based on each mutant's comparison, wherein the threshold biomass value is obtained from one or more members of a mutant population containing the one or more mutant cereal crop lines or is obtained from a non-mutagenized cereal crop line corresponding to the one or more mutant cereal crop lines.

A grain yield prediction can be any predictive label assigned to a given mutant line. For example, in some embodiments, a grain yield prediction can include a categorized probable yield prediction. For example, grain yield prediction categories can, in some embodiments, include ‘high yield’, ‘average yield’, or ‘low yield’, or ‘probable high yield’, ‘probable low yield’, or ‘probable above average yield’ and the like. Such categories can be assigned based on the one or more biomass values of the mutant line measured or estimated within the period of growth stages Z30 to Z39. For example, a mutant line having one or more biomass values in a top portion of biomass values of a plurality of mutant lines can, in some embodiments, be categorized as “probable high yield.” As another example, a mutant line having one or more biomass values exceeding a threshold biomass value (such as, e.g., a biomass value for a reference line), can, in some embodiments, be categorized as “probable high yield.” As another example, a mutant line having one or more biomass values in a bottom portion of biomass values of a plurality of mutant lines can, in some embodiments, be categorized as “probable low yield” or “probable average yield” depending on experimental set up. As another example, a mutant line having one or more biomass values below a threshold biomass value (such as, e.g., a biomass value for a reference line), can, in some embodiments, be categorized as “probable low yield.” As another example, a mutant line having one or more biomass values about the same as a threshold biomass value (such as, e.g., a biomass value for a reference line), can, in some embodiments, be categorized as “probable average yield.”

In some embodiments, generating a grain yield prediction can include using statistical analysis or modelling, including advanced statistical analysis or modelling, to predict the yield performance, make a selection of mutant lines, and the like. In some embodiments, a grain yield prediction can be assigned a value, for example, a numerical value, such as a value of likely grain per plant, grain per meter, or the like. In some embodiments, a grain yield prediction can be a relative prediction, such as, but not limited to a likelihood that a particular mutant line will produce a higher or lower yield than another mutant line.

In some embodiments, yield prediction or mutant line selection can further include identifying one or more traits other than biomass in each of the one or more mutant lines, and, optionally, can further comprise selecting one or more of the one or more mutant lines having a high biomass value for further use based on the one or more identified traits other than biomass. Exemplary non-limiting traits other than biomass can include ground cover, greenness, erectness, canopy temperature, vigor, winter-hardiness, and combinations thereof.

In another aspect, methods are provided for high throughput screening of mutant cereal crop lines. In some embodiments, the methods can comprise:
growing one or more mutant cereal crop lines;
estimating, by a LiDAR-based method for biomass estimation, on at least a portion of a test row, at least one biomass value for each mutant line within a period corresponding to Zadok's growth stages of from about Z30 to about Z39;
identifying one or more mutant lines having at least one high biomass value within the period, wherein high biomass value is selected from:
   a biomass value that is within a highest portion of biomass values for a plurality of mutant lines, wherein the biomass values for each of the plurality of mutant lines are measured or estimated at a same or similar growth stage within the period, wherein the plurality of mutant lines includes the one or more mutant cereal crop lines and wherein the plurality of mutant lines is a mutant population, or
   a biomass value exceeding a threshold biomass value, wherein the threshold biomass value is obtained from one or more members of a mutant population containing the one or more mutant cereal crop lines or is obtained from a non-mutagenized cereal crop line corresponding to the one or more mutant cereal crop lines.

In some embodiments, the methods can comprise:
growing one or more mutant cereal crop lines;
estimating, by a LiDAR-based method for biomass estimation, on at least a portion of a test row, at least one biomass value for each mutant line within a period corresponding to Zadok's growth stages of from about Z30 to about Z39;
generating a grain yield prediction for each mutant line based on the at least one biomass value for the mutant line.

The mutant lines can be screened for further use or processing based on the biomass value or the yield prediction. Generating a grain yield prediction for each mutant line can comprise at least one selected from: ordering the biomass values for each of at least two mutant lines belonging to a mutant population in an ascending or descending order of biomass values and assigning a relative yield prediction to each mutant line corresponding to a position of the mutant's at least one biomass value in the order of biomass values, wherein the biomass values for each mutant line is measured or estimated at a same or similar growth stage within the period; comparing the at least one biomass value for each mutant line with biomass values for a plurality of mutant lines, wherein the biomass values for each of the plurality of mutant lines are biomass values for each, and generating a yield prediction based on a relation of the mutant's at least one biomass value to the biomass values for each of the plurality of mutant lines, wherein the plurality of mutant lines includes the one or more mutant cereal crop lines and wherein the plurality of mutant lines is a mutant population; or determining a comparison for each mutant's biomass value against a threshold biomass value and generating a yield prediction based on each mutant's comparison, wherein the threshold biomass value is obtained from one or more members of a mutant population containing the one or more mutant cereal crop lines or is obtained from a non-mutagenized cereal crop line corresponding to the one or more mutant cereal crop lines.

While this disclosure contains many specific implementation details, these should not be construed as limitations on the scope of the subject matter or on the scope of what can be claimed, but rather as descriptions of features that can be specific to particular implementations. Certain features that are described in this disclosure in the context of separate implementations can also be implemented, in combination, in a single implementation. Conversely, various features that are described in the context of a single implementation can also be implemented in multiple implementations, separately, or in any suitable sub-combination. Moreover, although previously described features can be described as acting in certain combinations and even initially claimed as such, one or more features from a claimed combination can, in some cases, be excised from the combination, and the claimed combination can be directed to a sub-combination or variation of a sub-combination.

Particular embodiments of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations can be considered optional), to achieve desirable results.

Accordingly, the previously described example implementations do not define or constrain this disclosure. Other changes, substitutions, and alterations are also possible without departing from the spirit and scope of this disclosure.

### EXAMPLES

### Example 1

### Material and Set-Up in Season 1 Field Trials

Field trials were performed during Season 1 using seeds for a mutant population of the wheat variety “Mace”.

1750 different mutant lines were planted in a partial replicated field trial design.

Mutant lines were sown in a row plot setup similar to that shown in FIG. 2, with 4 different mutant lines per plot sown in a central portion of the plot and flanked with a border variety (Mace) at both ends. 50 seeds per mutant line were sown in pre-irrigated soil in a plot of 1.5 m with 20 cm distance between rows and 0.5 m distance between plots.

### In Season Analysis Season 1

LIDAR measurements of early biomass were performed on 7 different dates, corresponding to 7 different Zadok's growth stages, using a Lidar sensor mounted on a PHENOMOBILE Lite (Jimenez-Berni Jose A., et al., Front. Plant Sci., 27 Feb. 2018, Vol. 9, Art. 237). In addition, RGB pictures were taken, which were used to verify the developmental stage of the plant at the time of each Lidar analysis. Biomass of each row was determined by algorithmic analysis of the Lidar data. Data from one of the dates could not be analyzed.

### Post Harvest Analysis Season 1

Each row was harvested individually and the following parameters were measured: number of spikes per row and plant; number of seeds per row, plant and spike; grain weight per row (row yield), plant and spike and total grain weight (TGW). BLUE data were calculated for all parameters resulting in a single value per mutant line and parameter.

### Data Analysis Season 1

The correlation between grain weight per row (row yield) and early biomass measured by Lidar at different measurement dates is shown in Table 1.

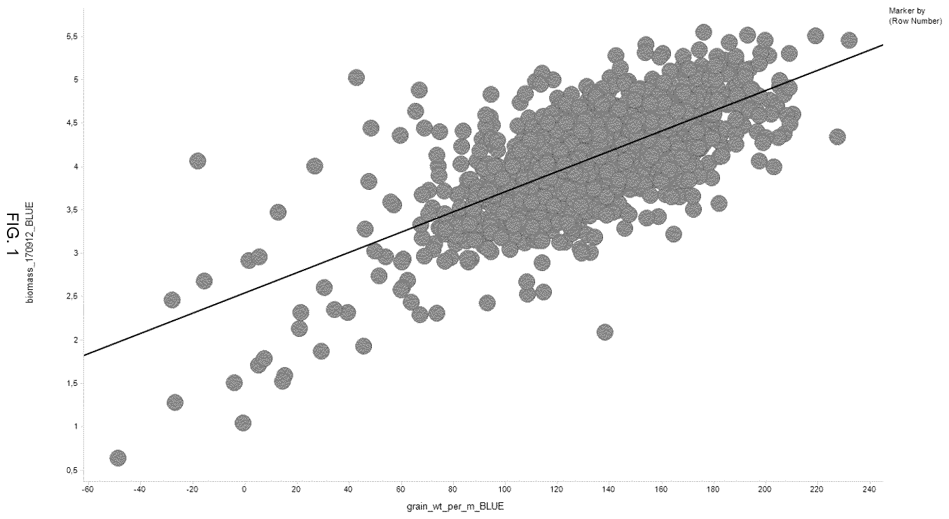

The correlation between row yield and row biomass at approximately Zadok's growth stage Z38 (Date 2) for all mutant lines (r²=0.461) is shown in FIG. 1.

TABLE 1

| Date of | r² for Correlation row |
|---|---|
| measurement | yield and biomass |
| Date 1 | 0.417 |
| Date 2 | 0.461 |
| Date 3 | 0.371 |
| Date 4 | 0.414 |
| Date 5 | 0.342 |
| Date 6 | 0.315 |

As analysis of the Lidar data was not completed in time, post-harvest data was used to select a set of 190 mutant lines based on improved yield components or combination of yield components for further field testing.

### Field Trials Season 2-Row Plots

Field trials in Season 2 followed the same set-up as in Season 1.

60 seeds per mutant were sown in a plot of 1.5 m with 20 cm distance between rows and 0.5 m distance between plots. Some reference lines were added in the trial. All 1750 mutants were sown in two replications.

### In Season Analysis Season 2

LIDAR measurements of early biomass were performed on 7 different dates, corresponding to 7 different Zadok's growth stages, using a Lidar sensor mounted on a PHENOMOBILE Lite (Jimenez-Berni Jose A., et al., Front. Plant Sci., 27 Feb. 2018, Vol. 9, Art. 237). In addition, RGB pictures were taken, which were used to verify the developmental stage of the plant at the time of each Lidar analysis. Biomass of each row was determined by algorithmic analysis of the Lidar data.

### Post Harvest Analysis Season 2

Each row was harvested individually individually and the following parameters were measured: number of spikes per row and plant; number of seeds per row, plant and spike; grain weight per row (row yield), plant and spike and total grain weight (TGW). BLUE data were calculated for all parameters resulting in a single value per mutant line and parameter.

### Data Analysis Season 2

The correlation between grain weight per row (row yield) and early biomass measured by Lidar at different measurement dates is shown in Table 2.

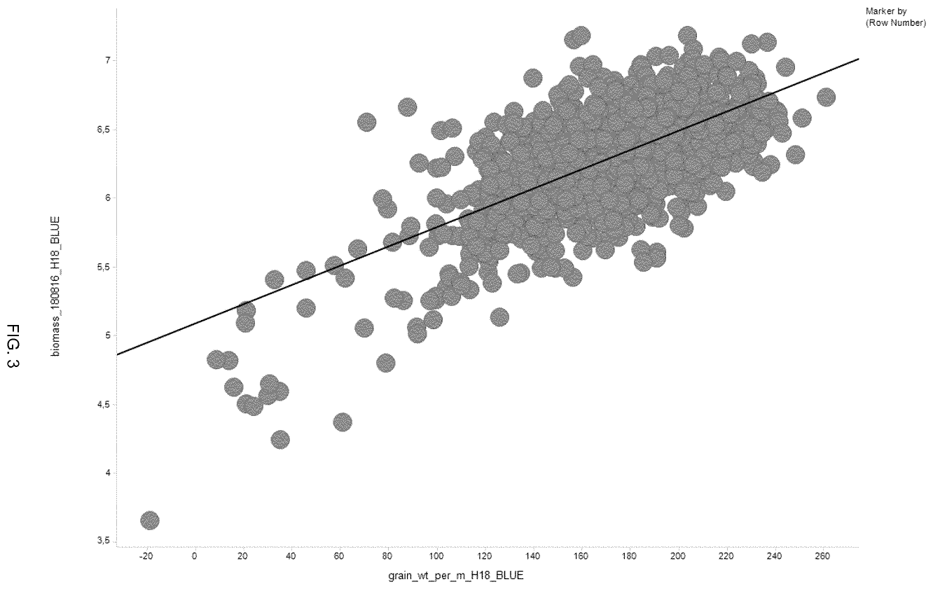

The correlation between row yield and row biomass at approximately Zadok's growth stage Z35 (Date 2) for all mutant lines (r²=0.422) is shown in FIG. 3.

TABLE 2

| Date of | r² for Correlation row |
|---|---|
| measurement | yield and biomass |
| Date 1 | 0.370 |
| Date 2 | 0.422 |
| Date 3 | 0.412 |
| Date 4 | 0.002 |
| Date 5 | 0.040 |
| Date 6 | 0.034 |

### Field Trials Season 2-Full Plots

Full-plot trials with 8 m² plots were performed to test the mutants selected in from Season 1. This trial, 190 selected mutants were sown in 2 plots/mutant line at a density of 250 grains/m² to achieve about 200 grains/m², a similar density to the row plots, using the grain weight estimations from Season 1 expressed as Thousand Kernel Weight.

Field trials were performed according to standard agronomical practices. Irrigation was applied to avoid drought stress. Plots were harvested and data for yield and yield components were generated and statistical analysis was performed to generate BLUE data.

### Across Season Analysis

### Row Plots Season 1

As analysis of the Lidar data was not completed in time, post-harvest data were used to select a set of 190 mutant lines based on improve row yield and/or improve yield components or combinations of improved yield components for further field testing.

From the 100 mutant lines selected for increased row yield (grain weight per row) 83 would also have been selected from LiDAR based row biomass at Z38, if 30% best performing biomass lines from this population were selected.

### Row Plots Season 1 vs Full Plots Season 2

From the 190 mutant lines tested in yield plots, 39 mutant lines performed at least 2% better than the un-mutagenized control and 21 mutant lines performed at least 4% better than the un-mutagenized control for yield.

From the 39 good performing lines, 30 mutant lines (76%) would have been selected based on early biomass at timepoint 2 (30% best lines), whereas only 23 mutant lines (59%) would have been selected based on Season 1 row yield.

From the 21 best performing lines, 16 mutant lines (76%) would have been selected based on early biomass at timepoint 2 (30% best lines), whereas only 13 mutant lines (62%) would have been selected based on Season 1 row yield.

This indicates that early biomass at a specific developmental stage is a better predictor for full plot yield than traditional row yield data.

1. A method comprising:
growing one or more mutant cereal crop lines;
measuring or estimating at least one biomass value for each mutant line within a period corresponding to Zadok's growth stages of from about Z30 to about Z39;
identifying one or more mutant lines having at least one high biomass value within the period, wherein high biomass value is selected from:
a biomass value that is within a highest portion of biomass values for a plurality of mutant lines, wherein the biomass values for each of the plurality of mutant lines are measured or estimated at a same or similar growth stage within the period, wherein the plurality of mutant lines includes the one or more mutant cereal crop lines and wherein the plurality of mutant lines is a mutant population, or a biomass value exceeding a threshold biomass value, wherein
the threshold biomass value is obtained from one or more members of a mutant population containing the one or more mutant cereal crop lines or is obtained from a non-mutagenized cereal crop line corresponding to the one or more mutant cereal crop lines.

2.-27. (canceled)

28. A method comprising:
growing one or more mutant cereal crop lines;
measuring or estimating at least one biomass value for each mutant line within a period corresponding to Zadok's growth stages of from about Z30 to about Z39;
generating a grain yield prediction for each mutant line based on the at least one biomass value for the mutant line, wherein generating comprises at least one selected from the following:
ordering the biomass values for each of at least two mutant lines belonging to a mutant population in an ascending or descending order of biomass values and assigning a relative yield prediction to each mutant line corresponding to a position of the mutant's at least one biomass value in the order of biomass values, wherein the biomass values for each mutant line is measured or estimated at a same or similar growth stage within the period;
comparing the at least one biomass value for each mutant line with biomass values for a plurality of mutant lines, wherein the biomass values for each of the plurality of mutant lines are measured or estimated at a same or similar growth stage within the period, and generating a yield prediction based on a relation of the mutant's at least one biomass value to the biomass values for each of the plurality of mutant lines, wherein the plurality of mutant lines includes the one or more mutant cereal crop lines and wherein the plurality of mutant lines is a mutant population; or
determining a comparison for each mutant's biomass value against a threshold biomass value and generating a yield prediction based on each mutant's comparison, wherein the threshold biomass value is obtained from one or more members of a mutant population containing the one or more mutant cereal crop lines or is obtained from a non-mutagenized cereal crop line corresponding to the one or more mutant cereal crop lines.

29. The method of claim 1, wherein the threshold biomass value is a biomass value of a reference crop line measured or estimated at a same or similar growth stage within the period, or an average or median biomass value determined from a plurality of mutant lines measured or estimated at a same or similar growth stage within the period.

30. The method of claim 1, further comprising selecting one or more of the one or more mutant lines having a high biomass value for further use.

31. The method of claim 30, wherein the further use is selected from future breeding, genotyping, yield trialing, harvest, genetic mapping, or combinations thereof.

32.-33. (canceled)

34. The method of claim 1, wherein measuring or estimating at least one biomass value for each mutant line comprises measuring or estimating at least one biomass value for a portion of a row, a plot, or a farmer's field containing plants of the mutant line.

35. The method of claim 34, wherein measuring or estimating at least one biomass value for each mutant line comprises measuring or estimating at least one biomass value for a portion of a test plot, wherein the test plot contains a single mutant line.

36. The method of claim 34, wherein the yield prediction is selected from a plot yield prediction or a farmer's field yield prediction.

37.-38. (canceled)

39. The method of claim 34, wherein measuring or estimating at least one biomass value for each mutant line comprises measuring or estimating at least one biomass value for a portion of a test row.

40. The method of claim 39, wherein the test row is one of a plurality of interplanted rows, wherein each row independently contains plants from a single mutant line or reference line, and wherein each row contains a different mutant line or reference line than an adjacent row.

41. The method of claim 39, wherein the test row is part of a row plot comprising a central portion containing one or more parallel adjacent test rows, wherein the central portion is flanked by at least one border row at each of two opposite sides of the central portion, wherein each of the at least one border rows are sown parallel to the test rows of the central portion.

42. The method of claim 41, wherein each test row of the central portion independently contains a mutant line and each border row independently contain a reference line.

43. The method of claim 39, wherein the growing one or more mutant lines comprises growing each mutant line in a single row.

44. (canceled)

45. The method of claim 34, wherein the yield prediction is selected from a row yield prediction, a plot yield prediction, or a farmer's field yield prediction.

46.-48. (canceled)

49. The method of claim 1, wherein measuring or estimating biomass is performed by a non-destructive measurement or estimation method and wherein the non-destructive measurement or estimation method is selected from a LiDAR-based method for biomass estimation and a NDVI-based method for biomass estimation.

50.-51. (canceled)

52. The method of claim 1, further comprising generating at least one of the one or more mutant cereal crop lines by mutagenesis.

53.-55. (canceled)

56. The method of claim 1 further comprising selecting one or more of the one or more mutant lines having a high biomass value for further use based on the identifying one or more traits other than biomass.

57. A method for high throughput screening of mutant cereal crop lines comprising the method of claim 49, wherein the mutant lines are screened for further use or processing based on the biomass value.

58. A method for high throughput screening of mutant cereal crop lines comprising the method of claim 49, wherein the mutant lines are screened for further use or processing based on the yield prediction.

59. A mutant cereal crop line produced by the method of claim 1.

60. The method of claim 52, wherein the mutagenesis is selected from chemical mutagenesis, physical mutagenesis, mutagenesis by genome editing, mutagenesis by transgenesis, or combinations thereof.
